(19) United States
(12) Patent Application Publication
(10) Pub. No.: US 2021/0321876 A1
(43) Pub. Date: Oct. 21, 2021

(54) SYSTEM AND METHOD FOR IMAGING, SEGMENTATION, TEMPORAL AND SPATIAL TRACKING, AND ANALYSIS OF VISIBLE AND INFRARED IMAGES OF OCULAR SURFACE AND EYE ADNEXA

(71) Applicants: Ehsan Zare Bidaki, Waterloo (CA); Paul J. Murphy, Waterloo (CA); Alexander Sheung Lai Wong, Waterloo (CA)

(72) Inventors: Ehsan Zare Bidaki, Waterloo (CA); Paul J. Murphy, Waterloo (CA); Alexander Sheung Lai Wong, Waterloo (CA)

(21) Appl. No.: 17/236,816

(22) Filed: Apr. 21, 2021

Related U.S. Application Data
(60) Provisional application No. 63/012,965, filed on Apr. 21, 2020.

(30) Foreign Application Priority Data
Apr. 21, 2020 (US) 63012965

(51) Int. Cl. A61B 5/01; G06T 5/50; G06T 7/11; G06T 7/00; A61B 3/14; A61B 5/11; A61B 3/113; A61B 3/10

(52) U.S. Cl. CPC A61B 5/015; G06T 5/50; G06T 7/11; G06T 7/0016; G06T 2207/30041; A61B 5/1128; A61B 3/113; A61B 3/101; G06T 2207/10048; A61B 3/14

(57) ABSTRACT

An automatic system and method for non-invasive imaging and identification of specific ocular structures of the eye and adnexa tissues by synchronous segmentation of visual and infrared images; that can produce spatial temperature profiles within each segmented area of the eye and adnexa; that can track eye and head movement and eye-blinks during the period of measurement to remove artefacts and maintain synchronicity; that can track ocular surface and eye adnexa temperature profiles over time; that can assist in diagnosis of eye disease; that can produce diagnostic indicators for ocular disease diagnosis and study of the eye. The system comprises infrared and visible light cameras for imaging the ocular structures, and a digital signal processing unit for processing the acquired infrared and visible images to output segmentations of the images for identification of different areas of the eye surface, including pupil, cornea, conjunctiva, and eyelids. The system further captures synchronous infrared and visible images from each segmented area of the ocular surface over the time of measurement. A digital signal processing unit processes and analyzes the infrared and visible images to generate descriptive outputs on temporal and spatial changes in the infrared and visible images over the time of measurement, as well as produce diagnostic indicators for ocular disease diagnosis and study of the eye.

### CROSS-REFERENCE TO RELATED APPLICATIONS

This application claims priority from U.S. Provisional Application No. 63/012,965 filed on Apr. 21, 2020, which is incorporated by reference herein in its entirely.

### FIELD OF THE INVENTION

The present disclosure relates to a system and method for imaging and analysis of ocular surfaces for ocular diseases and studies of the eye.

### BACKGROUND

Body temperature reflects physiological information about human health. It can be assessed using an invasive (contact) method, e.g. thermal expansion of a liquid, or a non-invasive (non-contact) method, e.g. IR imaging (thermography). IR imaging has many advantages over contact methods: it is non-invasive and so does not alter the target tissue structure or stability which would alter the temperature profile, can be obtained in real-time, can provide continuous data over the time period of measurement, can provide data over a large surface area, and is very accurate. Clinically, IR imaging is used to observe areas of inflammation in the body. Inflammation is produced as part of the body's response to infection. It has four characteristics: swelling, redness, pain, and heat. IR thermography captures images of the heat response from inflammation and can be used as part of a diagnosis assessment or to monitor infection and/or treatment progress. A precise IR thermogram (temperature profile image) can help physicians to diagnose infections and diseases of the eye with much-improved precision.

IR imaging has been used to monitor temperature changes over the surface of the eye. Published papers have reported the results of studies looking at temperature changes associated with ocular infections and disease diagnosis, as well as changes in tear-film structure between blinks and during contact lens wear.

Ocular thermography enables analysis of the tear-film without disrupting the structure. The tear film is a dynamic structure, with variable thickness and composition. The tear-film plays several important roles in the eye, including lubrication, nutrition, and protection from foreign bodies. The outer layer of the tear-film is a lipid layer which maintains tear-film stability and prevents excessive evaporation. Under normal conditions, the tear-film layer undergoes a repeated cycle of formation, destabilization, break-up, and reformation. Since the tear film is inherently unstable, variations in evaporation across the surface is a natural phenomenon, which may have a role in triggering a blink or in detecting changes in local ambient environmental conditions. Reformation occurs by the action of the eyelids during a blink cycle, and the time between a blink and tear-film break-up is named tear-film break-up time (TBUT). TBUT is monitored clinically to provide a measure of the quality of the tear-film. Tear-film instability will affect ocular surface temperature (OST) by increasing the level of evaporation from the surface and is one of the key factors in dry eye disease. Thus, OST can be used for TBUT measurement. The rate of change or the size of change or the variation in OST across the ocular surface is greater in dry eye patients. These effects can be observed using IR thermography.

Previous methods for IR imaging of the eye surface can be grouped into single-camera and dual-camera methods. Single camera systems only use an IR thermal camera. They are aligned manually and assign the point or area of interest manually. They lack resolution in the IR image, and the image cannot be segmented, eye movement cannot be tracked, and artefacts from eyelid blinking cannot be removed. Dual-camera systems use a combination of an IR and visible light camera. They are aligned manually and assign the point or area of interest manually. They use the visible image to find the corneal boundary in the thermal image, but do not attempt image segmentation. They do not track eye movements or remove eyelid blinking artefacts.

US patent application no. 2008/0174733A1 describes a dual (IR and visible light camera) combination for diagnosing dry eye disease. A visible light camera was installed on top of the IR thermal camera to assist the operator manually in locating the cornea in the IR image. The field of view of the visible light camera is aligned to match the field of view of the IR thermal camera. The system incorporated a point of fixation for the subject, and the operator moved the imaging cameras to locate the cornea in the centre of the IR image. The visible light camera and a mirrored reflector were used to help the subject adjust their head position to bring the area of interest into the centre of the visual image, and thus the IR image. Points of interest on the corneal surface are manually selected by the operator within the IR image, and data recorded from these pixel points over a period of measurement. The change in temperature of each pixel point was presented to the operator as a line graph showing temperature against time.

US patent application no. 2015/0342465 proposed a single camera method for calibrating the measurement of surface temperature of a black body comprising an IR thermal camera and a contact sensor to measure the black body temperature. This method assists in calibration of the IR thermal camera, and in accounting for possible temperature drift in the IR thermal camera during the period of measurement. However, it has significant limitations.

US patent application no. 2017/0347890A1 describes a portable device for measuring eye temperature. This device is a multiple camera version of a single camera method. The device comprises temperature sensors, a signal processing unit, and a transceiver. The transceiver receives the temperature signals and sends them wirelessly to a mobile device for further processing. The temperature sensors are mounted in a wearable visor with embedded wireless sensors that are directed towards the eye. Each sensor measures the OST from a single point on the ocular surface. The device produces a series of single point measurements from the surface of the eye, with one measurement associated with one sensor.

Some other groups using dual IR and visible light camera systems, attempted to manually overlap the images to assist in locating the point or area of interest in the IR image. However, the image adjustment is highly dependent on the camera position and camera specification. For all of these methods, the point or areas of interest are manually selected by the operator within the IR image. These limitations make such a system impractical and infeasible to use for imaging and analysis of ocular surfaces for ocular disease and studies of the eye in practical scenarios, especially real-time, high-speed scenarios, given the time consuming, manual nature of such a configuration and is prone to error.

Kamao et al. (2011) described a method for measuring eye temperature using IR and visible light cameras embedded in a single device. The field of view of the visible light camera is aligned to match the field of view of the IR thermal camera. In this configuration, the corneal boundary of the subject's eye could be identified with improved accuracy, but the two cameras were not synchronized and so is impractical and infeasible to use for imaging and analysis of ocular surfaces for ocular disease and studies of the eye in practical scenarios, especially real-time, high-speed scenarios, given the need for manual processing of collected data and is also prone to error.

Su et al. (2014) described a dual camera method of an IR thermal camera and a visible light camera. A Germanium mirrored filter was placed in the IR optical pathway to reflect visible light to a visible light camera to overlap the two images. The frame rate was set at 30 frames per second. Post-hoc processing of the IR and visible light camera images identified matching areas of temperature or colour change, but OST was not reported.

Li et al. (2015) described a dual camera method of an IR thermal camera and a visible light camera. Images from each camera were time-synchronized, but not registered together. Segmentation of the images was not attempted. Post-hoc processing of the IR and visible light camera images identified matching areas of temperature or colour change. OST change was reported for a manually selected area of interest only.

Kricancic et al. (2017) described a dual camera method of an IR thermal camera and a visible light camera. A Germanium mirror was placed in the IR optical pathway to reflect visible light to a visible light camera to overlap the images. Post-hoc processing of the IR and visible light camera images identified matching areas of temperature or colour change. OST change was reported for a manually selected area of interest only.

Each of the above references has various limitations which may inhibit full realization of imaging and analysis techniques. Therefore, what is needed is an improved system and method which addresses at least some of these limitations in the prior art.

### SUMMARY OF THE INVENTION

The present disclosure relates to a system and method for imaging and analysis of ocular disease and studies of the eye. More generally, the present system and method provides an improved system and method for automatically and non-invasively imaging the ocular surface and adnexa tissues using infrared (IR) thermal cameras and visible light cameras synchronously. In an embodiment, the present system and method segments the images produced to identify specific ocular structures and measures ocular surface temperature (OST) within segmented areas by tracking the OST precisely, including by monitoring eye tracking and eye blinking during measurement to remove artefacts and maintain synchronicity. Temporal and spatial changes in the IR and visible images are tracked over time, and diagnostic indicators for ocular disease diagnosis and study of the eye are produced.

In various aspects, the present system and method locates specific eye locations in the thermogram; removes artefacts produced by eye and head movements; removes artefacts produced by eyelid blinking; gathers and analyses all data pixel points within the area of interest; outputs segmentations of the images for identification of different areas of the eye surface, including pupil, cornea, conjunctiva, and eyelids; generates descriptive outputs on temporal and spatial changes in the infrared (IR) and visible images over the time of measurement; and produces diagnostic indicators for ocular disease diagnosis and study of the eye.

In an illustrative embodiment, the present system and method may comprise one or more IR thermal cameras and one or more visible light cameras installed on a camera mount in close proximity to each other. Using more than one of each type of camera can, but not limited to: enable higher temporal resolution with temporally offset measurements, enable spatial resolution with spatially offset measurements, and enable three-dimensional measurements from multiple views. Note that a camera may include both visible light sensors and IR sensors for a more compact form factor.

In an embodiment, the cameras are mounted horizontally with respect to each other. The camera mount is fixed to the top of a vertical pillar or support that is installed on a movable base. The movable base can be moved in the x/y/z planes by the operator. This arrangement enables the camera mount to be moved to align the cameras with the subject's eye and to focus the image plans of the cameras on the subject's eye and adnexa tissues.

In an embodiment, the system and method further comprises a separate head-rest and chin-rest positioned in front of the camera mount on which the subject rests their head during measurements.

In another embodiment, the system and method further comprises a digital signal processing unit that registers and synchronizes imaging data from IR and visible light camera image sequences.

In another embodiment, the system and method further comprises a digital signal processing unit that generates segmented areas of interest within the IR and visible light camera images.

In another embodiment, the system and method further comprises a digital signal processing unit that processes IR and visible images and produces segmentations of pupil, cornea, conjunctiva, eyelids, and areas within these regions, in the visible images of the subject's eye.

In another embodiment, a digital signal processing unit that processes IR and visible images and registers the IR and visible image sequences for precise localization of the segmented pupil, cornea, conjunctiva, eyelids, and areas within these regions, from the visible images to the IR images of the subject's eye.

In another embodiment, a digital signal processing unit that processes IR and visible images and detects subject eye movements and tracks the area of interest in the visible images over the period of measurement.

In another embodiment, a digital signal processing unit that processes IR and visible images and detects subject eyelid blinks and remove any artefacts affecting the area of interest in the images over the period of measurement.

In another embodiment, a digital signal processing unit that processes and analyses the IR and visible light camera data streams per pixel for temperature, texture, and colour components.

In another embodiment, a digital signal processing unit that processes and analyses the IR and visible light camera data streams and outputs spatial temperature profiles across the area of interest of the subject's eye to produce three-dimensional plots of temperature change.

In another embodiment, a digital signal processing unit that processes and analyses the IR and visible light camera data streams and outputs temporal temperature profiles across the area of interest of the subject's eye and over the period of measurement to produce plots of temperature changes.

In another embodiment, a digital signal processing unit that processes and analyses the IR and visible light camera data streams and outputs texture and colour change profiles across the area of interest of the subject's eye over the period of measurement.

In another embodiment, a digital signal processing unit that processes and analyses the IR and visible light camera data streams and outputs descriptors of temperature change, texture change and colour change associated with ocular surface evaporation, ocular surface esthesiometry, tear break-up time, contact lens wear, computer vision syndrome, infection and disease of the eye and ocular adnexa.

In this respect, before explaining at least one embodiment of the system and method of the present disclosure in detail, it is to be understood that the present system and method is not limited in its application to the details of construction and to the arrangements of the components set forth in the following description or illustrated in the drawings. The present system and method is capable of other embodiments and of being practiced and carried out in various ways. Also, it is to be understood that the phraseology and terminology employed herein are for the purpose of description and should not be regarded as limiting.

### BRIEF DESCRIPTION OF DRAWING

FIG. 1 shows a schematic view of the whole system in accordance with an illustrative embodiment.

FIG. 2 shows an illustrative frontal view of a subject's eye surface and adnexa that can be localized in the system for temperature profile.

FIG. 3 shows an example of ocular surface segmentation in accordance with an illustrative embodiment.

FIG. 4 demonstrates the function of each DSP module or unit and the output of each unit with sample video files recorded by the system in accordance with an illustrative embodiment.

FIG. 5 shows a schematic diagram of a computer system which may provide an operating environment for one or more embodiments of the present system and method.

### DETAILED DESCRIPTION

As noted above, the present disclosure relates to a system and method for imaging and analysis of ocular disease and studies of the eye. More generally, the present system and method provides an improved system and method for automatically and non-invasively imaging the ocular surface and adnexa tissues using infrared (IR) thermal cameras and visible light cameras synchronously. In an embodiment, the present system and method segments the images produced to identify specific ocular structures and measures ocular surface temperature (OST) within segmented areas by tracking the OST precisely, including by monitoring eye tracking and eye blinking during measurement to remove artefacts and maintain synchronicity. Temporal and spatial changes in the IR and visible images are tracked over time, and diagnostic indicators for ocular disease diagnosis and study of the eye are produced.

A key requirement for ocular surface thermography is the ability to locate the corneal area in the thermogram. However, conduction of heat within the eye ensures that the thermal profile imaged by an IR thermal camera of the ocular surface describes an unfocused thermogram that does not match the underlying anatomical features. This makes it difficult to precisely locate an area of interest on the ocular surface using only the IR image. The majority of existing camera systems described in the background lack a method for detecting the corneal boundary, corneal centre, and conjunctiva, and all existing methods for identifying the point or area of interest in the eye require the input from the operator to manually select the point or area of interest.

A second requirement is the ability to consistently measure from the same location on the ocular surface. Current methods for IR imaging of the eye incorporate a fixation target for the subject to view. However, during steady fixation by the eye on a point of interest, small eye and head movements still occur. These movements cause relative movements in the areas of interest on the eye and ocular surface during the period of measurement which degrade the accuracy of measurement over the period of measurement. No current methods for IR imaging of the eye incorporate an eye-tracking ability to counteract the effects of eye and head movement.

A third requirement is the ability to track changes in the ocular surface temperature over a period of time without the effect of artefacts from eyelid blinking. The eyelid covers the area of interest on the ocular surface and introduces an artefact in the temporal temperature profile. All current systems are able to record temporal changes for the selected point or area of interest, but in the prior art data collected must be manually screened for blinking artefacts. No current systems for IR imaging of the eye incorporate an automatic method for removing eyelid blinking artefacts.

A fourth requirement is to be able to collect and analyse temperature data from all pixel points across the ocular surface within the image frame over the period of measurement. Current methods for IR imaging of the eye collect data from all pixels for image display, but select only a single pixel data point, multiple single pixel data points, or a described area of the surface for image analysis. No current system for IR imaging of the eye is able to report from all pixel points concurrently for data analysis.

A final requirement is that all of the four previously listed requirements should be completed automatically. No current system for IR imaging of the eye is able to automatically complete any of the four requirements.

The present system and method addresses at least some of these limitations.

In various aspects, the present system and method locates specific eye points of areas of interest in the thermogram; removes artefacts produced by eye and head movements; removes artefacts produced by eyelid blinking; gathers and analyses all data pixel points within the area of interest; outputs segmentations of the images for identification of different areas of the eye surface, including pupil, cornea, conjunctiva, and eyelids; generates descriptive outputs on temporal and spatial changes in the IR and visible images over the time of measurement; and produces diagnostic indicators for ocular disease diagnosis and study of the eye.

In an illustrative embodiment, the present system and method comprises one or more IR thermal cameras and one or more visible light cameras installed on a camera mount in close proximity to each other. The camera mount is fixed to a second mount installed on a movable base. The movable base can be moved in the x/y/z planes by the operator. This arrangement enables the camera mount to be moved to align the cameras with the subject's eye and to focus the image plans of the cameras on the subject's eye and adnexa tissues.

In an embodiment, the system and method further comprises a separate head-rest and chin-rest positioned in front of the camera mount on which the subject rests their head during measurements.

In another embodiment, the system and method further comprises a digital signal processing unit that registers and synchronises imaging data from IR and visible light camera image sequences.

In another embodiment, the system and method further comprises a digital signal processing unit that generates segmented areas of interest within the IR and visible light camera images.

In another embodiment, the system and method further comprises a digital signal processing unit that processes IR and visible images and produces segmentations of pupil, cornea, conjunctiva, eyelids, and areas within these regions, in the visible images of the subject's eye.

In another embodiment, a digital signal processing unit that processes IR and visible images and registers the IR and visible image sequences for precise localisation of the segmented pupil, cornea, conjunctiva, eyelids, and areas within these regions, from the visible images to the IR images of the subject's eye.

In another embodiment, a digital signal processing unit that processes IR and visible images and detects subject eye movements and tracks the area of interest in the visible images over the period of measurement.

In another embodiment, a digital signal processing unit that processes IR and visible images and detects subject eyelid blinks and removes any artefacts affecting the area of interest in the images over the period of measurement.

In another embodiment, a digital signal processing unit that processes and analyses the IR and visible light camera data streams per pixel for temperature, texture, and colour components.

In another embodiment, a digital signal processing unit that processes and analyses the IR and visible light camera data streams and outputs spatial temperature profiles across the area of interest of the subject's eye to produce three-dimensional plots of temperature change.

In another embodiment, a digital signal processing unit that processes and analyses the IR and visible light camera data streams and outputs temporal temperature profiles across the area of interest of the subject's eye and over the period of measurement to produce plots of temperature changes.

In another embodiment, a digital signal processing unit that processes and analyses the IR and visible light camera data streams and outputs texture and colour change profiles across the area of interest of the subject's eye over the period of measurement.

In another embodiment, a digital signal processing unit that processes and analyses the IR and visible light camera data streams and outputs descriptors of temperature change, texture change and colour change associated with ocular surface evaporation, ocular surface esthesiometry, tear break-up time, contact lens wear, computer vision syndrome, infection and disease of the eye and ocular adnexa.

Various illustrative embodiments of the present system and method will now be described in more detail with reference to the figures.

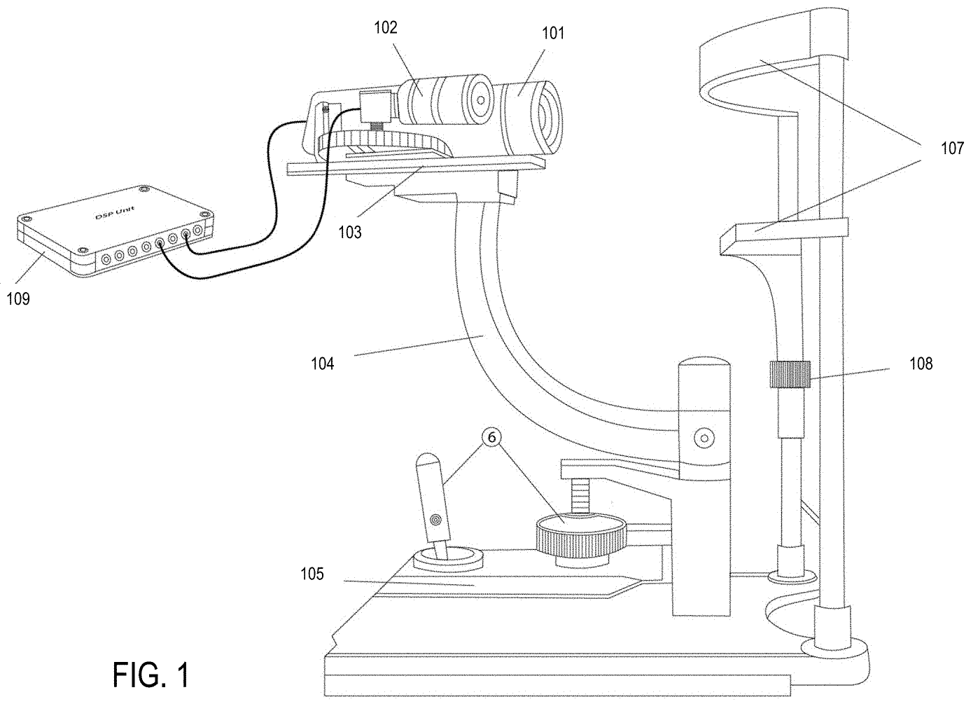

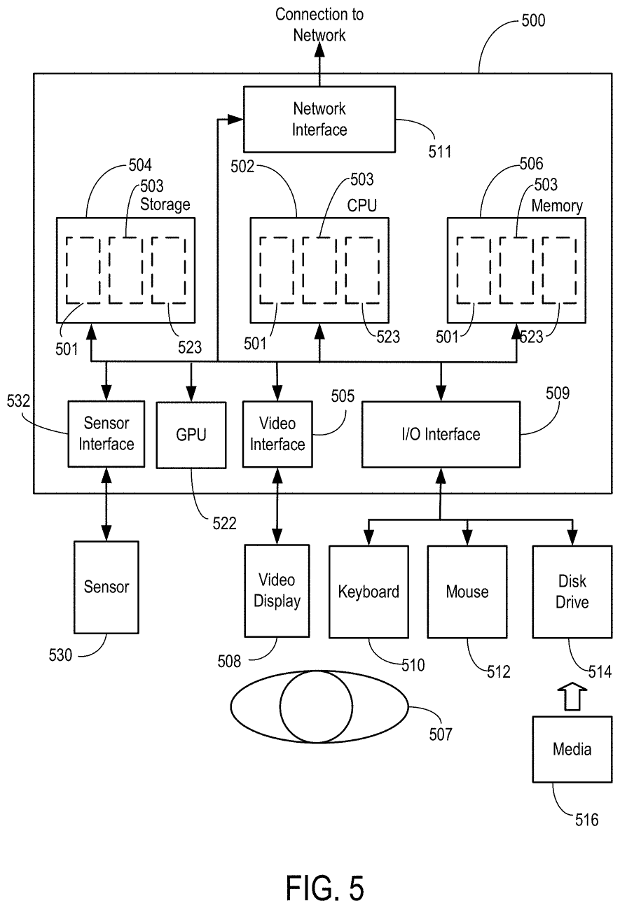

Now referring to FIG. 1, shown is a system in accordance with an illustrative embodiment. The system of FIG. 1 includes an IR thermal camera 101 to record thermal sequences from the subject's eye surface, a visible light camera 102 to record visible sequences, a camera mount 103 for camera installation, a vertical pillar 104 attached to a moveable base 105, and adjustment handles 106 for moving the camera system in front of the patient eyes, a head-rest and chin-rest 107 positioned in front of the camera mount on which the subject rests their head, a chin-rest height adjuster 108, and a digital signal processing (DSP) module or module or unit 109 for camera management and data analysis. One or more DSP modules or units 109 may be embodied, for example, by one or more computing devices as shown in FIG. 5 and described further below.

Still referring to FIG. 1, the system can capture both IR and visible image sequences from the surface of the subject's eye synchronously. The two cameras 101, 102 are aligned in such a way as to have the same field of view, and the DSP module or unit 109 registers the separate images from each camera together. As noted above, one or more IR thermal cameras and one or more visible light cameras may be installed on a camera mount in close proximity to each other. However, in alternative embodiments, the two cameras may be replaced by a single camera with one or more sensors, or replaced by a plurality of additional cameras to capture additional points of view. In this illustrative example, the camera mount 103 is positioned on top of the vertical pillar 104 that is attached to the movable base 105. The camera mount 103 is designed in a way that adjusts the relative position of the cameras forwards/backwards, up/down, left/right, and turning of the cameras in different angles of photography. The movable base 105 is designed to move forwards/backwards, up/down, and left/right to align the IR 101 and visible 102 cameras in front of the subject's eye.

In an embodiment, the cameras 102 and 103 are selected to capture images of a subject's eye or eyes at a sufficiently high resolution and at sufficiently high frame rates so as to capture clear, sharp images for processing. For example, camera image sensor resolutions of about 2MP or higher may be captured at high frame rates, or video images captured at 720p, 1080p, 4K or even higher resolutions at various frame rates may be utilized as may be required.

The DSP module or unit 109 is able to record both thermal and visible sequences synchronously. The recorded video files are time-coded and saved in a local disk for further analysis with the DSP module or unit 109. With a sufficiently high level of quality and sufficiently high frame rates for the captured images, the present system and method is able to process the images virtually in real-time.

In an embodiment, the DSP module or unit 109 processes the IR and visible sequence data to perform image processing and image analysis. In an initial step, the videos are overlaid using image registration techniques in the DSP module or unit 109 for further processing. In a subsequent step, the visible images are used to localize and segment specific parts of the eye including pupil, cornea, conjunctiva, and eyelids in the images, using image segmentation algorithms.

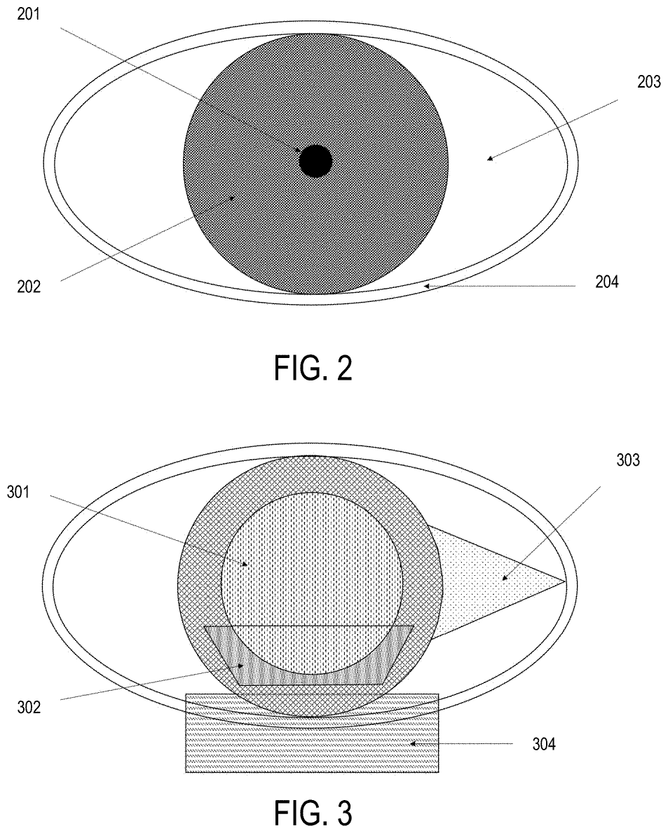

Now referring to FIG. 2, shown is an illustrative frontal view of a subject's eye surface and adnexa that can be localized in the system for temperature profile. The ocular surface parts and adnexa including pupil 201, iris 202, conjunctiva 203, and eyelid margin 204.

Now referring to FIG. 3, shown is an example of ocular surface segmentation in accordance with an illustrative embodiment. More specifically, ocular surface segmentation may include a central cornea sector 301, an inferior cornea sector 302, a conjunctiva sector 303, and an inferior eyelid margin sector 304.

In an embodiment, the segmented areas in the visible images are identified in the IR images.

In another embodiment, eye movements are detected in the synchronized images, and movement in the segmented areas identified and tracked. The dual camera system is synchronized by hardware triggering of the visible and thermal cameras under digital signal processing unit control. A further digital signal processing unit synchronizes the IR image files with the visible image files.

In another embodiment, eyelid blinking is identified in the synchronized images, and resulting artefacts removed from the image sequences. Semantic segmentation is used for corneal segmentation under digital signal processing unit control. The presence of a blink artefact is determined by monitoring the presence of the cornea in each visible light camera frame. The absence of a corneal segmentation indicates the presence of the eyelid, and the frame is detected as a containing a blink and removed from analysis.

In another embodiment, pixel characteristics from the IR and visible light camera images are analyzed over time to produce temperature, texture and colour profiles and rates of change across the area of interest of the subject's eye. Thermal data from the IR camera images and red/blue/green and grayscale data from the visible camera images for each frame of the recorded sequence is extracted for each pixel contained with the segmented area under observation. The video sequences for the IR and visible light camera images are recorded for storage. In an embodiment, a digital signal processing unit analyzes each frame from each video sequence to identify regions or profiles of thermal or texture change and to identify temporal and spatial changes in these regions or profiles over time. Presentation of this analysis is provided to the user in the form of statistical analyses that describe the thermal or textural characteristics of the segmented area. In an embodiment, analysis is completed after data collection, but may also be performed in real-time as the images are captured.

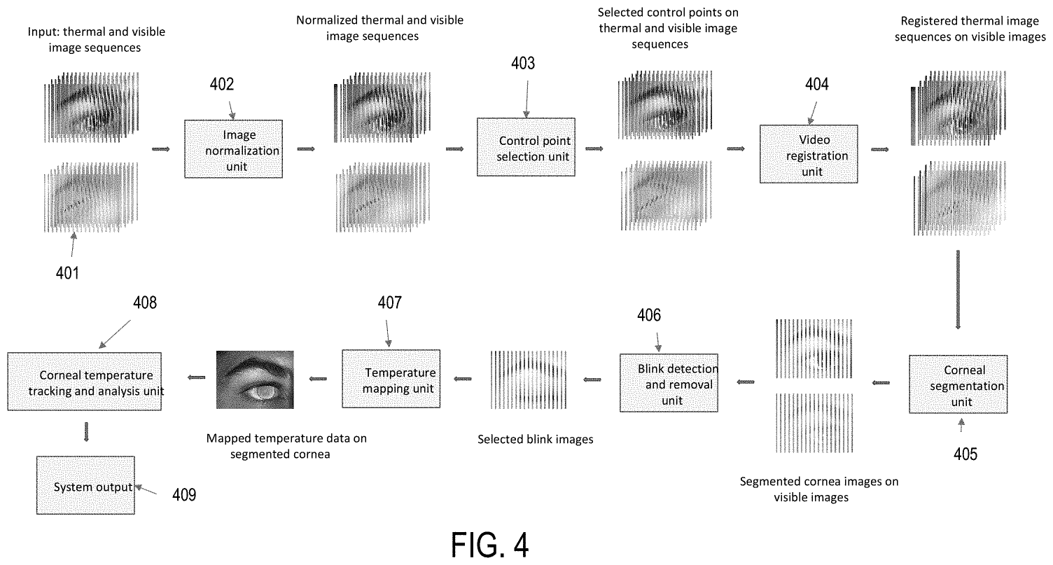

FIG. 4 demonstrates the function of each DSP module or unit and the output of each unit with sample video files recorded by the system in accordance with an illustrative embodiment using a dual camera setup for segmentation, tracking, and extracting temperature data of the cornea. As shown, the function of each DSP module or unit 109 and the output of each unit with sample video files recorded by the dual camera system is described in the figure. The IR and visible image sequences can be used as the input of the system 401. The image normalization unit 402 removes lens distortion from the image sequences. The undistorted image sequences are used as an input for the control point selection unit 403. The corresponding points on the first frames are selected. The corresponding points are localized on all subsequent frames of each camera's image sequence using an optical flow algorithm. The selected points and the normalized image sequences are used as an input for the video registration unit 404. The video registration unit registers the video files frame by frame using the control points. The visible video output file is used in the corneal segmentation unit 405. The cornea is segmented from the visible light camera image sequence using a semantic segmentation method. The corresponding corneal area in the IR image sequence is identified in the corneal segmentation unit. The blink frames are recognized from the image sequences and removed from the video files 406. The segmented IR image is mapped onto the visible image using the temperature mapping unit 407. The temperature of the corneal segment is tracked on the IR image sequence and extracted from each whole frame. Data analysis methods are used on the segmented IR data in the temperature mapping unit 408 and reported as the system output 409.

Advantageously, the output of the present system and method provides data on the localization of eye parts as the area of interest, and descriptive outputs data on temporal and spatial changes in ocular surface temperature (OST) over the area of interest.

Now referring to FIG. 5, the present system and method may be practiced in various embodiments. A suitably configured computer device, and associated communications networks, devices, software and firmware may provide a platform for enabling one or more embodiments as described above. By way of example, FIG. 5 shows a computer device 500 that may include a central processing unit (“CPU”) 502 connected to a storage unit 504 and to a random-access memory 506. The CPU 502 may process an operating system 501, application program 503, and data 523. The operating system 501, application program 503, and data 523 may be stored in storage unit 504 and loaded into memory 506, as may be required. Computer device 500 may further include a graphics processing unit (GPU) 522 which is operatively connected to CPU 502 and to memory 506 to offload intensive image processing calculations from CPU 502 and run these calculations in parallel with CPU 502. An operator 507 may interact with the computer device 500 using a video display 508 connected by a video interface 505, and various input/output devices such as a keyboard 510, pointer 512, and storage 514 connected by an I/O interface 509. In known manner, the pointer 512 may be configured to control movement of a cursor or pointer icon in the video display 508, and to operate various graphical user interface (GUI) controls appearing in the video display 508. The computer device 500 may form part of a network via a network interface 511, allowing the computer device 500 to communicate with other suitably configured data processing systems (not shown). It will be appreciated that computer device 500 may also be implemented in any number of different configurations, including as dedicated application-specific integrated circuits (ASIC) or chips integrated into the system.

Thus, in an aspect, there is disclosed a system for measuring ocular surface temperature, comprising: one or more cameras adapted to capture an infrared (IR) thermal image and a visible light image of an ocular surface; a camera positioning controller for controlling the one or more cameras to automatically capture synchronous IR and visible light images of multiple segmented areas of the ocular surface; and one or more digital processing modules adapted to: process the captured IR and visible light images to measure the ocular surface temperature (OST) of each segmented area over time; monitor at least one of head movement, eye tracking and eye blinking during OST measurement; and identify and remove artefacts in the visible image frame and the corresponding IR thermal image frame to maintain synchronicity of the images and obtain a more accurate OST measurement.

In an embodiment, the one or more digital processing modules is further adapted to segment the images produced to identify specific ocular structures and track OST temperatures precisely for those identified ocular structures.

In another embodiment, the temporal and spatial changes in the IR and visible images in a specific identified ocular structure is tracked in real-time.

In another embodiment, the temporal and special changes of the specific identified ocular structure and tracked OST temperatures are utilized as diagnostic indicators for ocular disease diagnosis and progression, regression, or remission.

In another embodiment, the specific identified ocular structures include pupil, iris, conjunctiva, and eyelids.

In another embodiment, the temporal and structural changes of the pupil, iris, conjunctiva, and eyelids are tracked over a period of time.

In another embodiment, the one or more digital processing modules are further adapted to measure tear-film dynamic assessment and instability of the eye.

In another embodiment, the one or more digital processing modules are further adapted to non-invasively measure tear-film break-up time (TBUT).

In another embodiment, the one or more digital processing modules are adapted to non-invasively measure and diagnose eye inflammation.

In another embodiment, the one or more digital processing modules are adapted to non-invasively measure and diagnose dry eye.

In another aspect, there is provided a method of measuring ocular surface temperature, comprising: providing one or more cameras adapted to capture an infrared (IR) thermal image and a visible light image of an ocular surface; providing a camera positioning controller for controlling the one or more cameras to automatically capture synchronous IR and visible light images of multiple segmented areas of the ocular surface; utilizing one or more digital processing modules: processing the captured IR and visible light images to measure the ocular surface temperature (OST) of each segmented area over time; monitoring at least one of head movement, eye tracking and eye blinking during OST measurement; and identifying and removing artefacts in the visible image frame and the corresponding IR thermal image frame to maintain synchronicity of the images and obtain a more accurate OST measurement.

In an embodiment, the method further comprises segmenting the images produced to identify specific ocular structures and track OST temperatures precisely for those identified ocular structures.

In another embodiment, the method further comprises tracking the temporal and spatial changes in the IR and visible images in a specific identified ocular structure in real-time.

In another embodiment, the method further comprises identifying the temporal and special changes of the specific identified ocular structure and utilizing the tracked OST temperatures as diagnostic indicators for ocular disease diagnosis and progression, regression, or remission.

In another embodiment, the specific identified ocular structures include pupil, iris, conjunctiva, and eyelids.

In another embodiment, the temporal and structural changes of the pupil, iris, conjunctiva, and eyelids are tracked over a period of time.

In another embodiment, the method further comprises utilizing one or more digital processing modules to measure tear-film dynamic assessment and instability of the eye.

In another embodiment, the method further comprises utilizing one or more digital processing modules to measure tear-film break-up time (TBUT).

In another embodiment, the method further comprises utilizing one or more digital processing modules to measure and diagnose eye inflammation.

In another embodiment, the method further comprises utilizing one or more digital processing modules to measure and diagnose dry eye.

While illustrative embodiments have been described, the scope of the invention is defined by the following claims.

1. A system for measuring ocular surface temperature, comprising:
one or more cameras adapted to capture an infrared (IR) thermal image and a visible light image of an ocular surface;
a camera positioning controller for controlling the one or more cameras to automatically capture synchronous IR and visible light images of multiple segmented areas of the ocular surface; and
one or more digital processing modules adapted to:
process the captured IR and visible light images to measure the ocular surface temperature (OST) of each segmented area over time;
monitor eye tracking and eye blinking during OST measurement; and
identify and remove artefacts in the visible image frame and the corresponding IR thermal image frame to maintain synchronicity of the images and obtain a more accurate OST measurement.

2. The system of claim 1, wherein the one or more digital processing modules is further adapted to segment the images produced to identify specific ocular structures and track OST temperatures precisely for those identified ocular structures.

3. The system of claim 2, where the temporal and spatial changes in the IR and visible images in a specific identified ocular structure is tracked in real-time.

4. The system of claim 3, wherein the temporal and special changes of the specific identified ocular structure and tracked OST temperatures are utilized as diagnostic indicators for ocular disease diagnosis and progression, regression, or remission.

5. The system of claim 3, wherein the specific identified ocular structures include pupil, iris, conjunctiva, and eyelids.

6. The system of claim 5, wherein the temporal and structural changes of the pupil, iris, conjunctiva, and eyelids are tracked over a period of time.

7. The system of claim 1, wherein the one or more digital processing modules are further adapted to measure tear-film dynamic assessment and instability of the eye.

8. The system of claim 1, wherein the one or more digital processing modules are further adapted to non-invasively measure tear-film break-up time (TBUT).

9. The system of claim 1, wherein the one or more digital processing modules are adapted to non-invasively measure and diagnose eye inflammation.

10. The system of claim 1, wherein the one or more digital processing modules are adapted to non-invasively measure and diagnose dry eye.

11. A method of measuring ocular surface temperature, comprising:
providing one or more cameras adapted to capture an infrared (IR) thermal image and a visible light image of an ocular surface;
providing a camera positioning controller for controlling the one or more cameras to automatically capture synchronous IR and visible light images of multiple segmented areas of the ocular surface; and
utilizing one or more digital processing modules:
processing the captured IR and visible light images to measure the ocular surface temperature (OST) of each segmented area over time;
monitoring eye tracking and eye blinking during OST measurement; and
identifying and removing artefacts in the visible image frame and the corresponding IR thermal image frame to maintain synchronicity of the images and obtain a more accurate OST measurement.

12. The method of claim 11, further comprising segmenting the images produced to identify specific ocular structures and track OST temperatures precisely for those identified ocular structures.

13. The method of claim 12, further comprising tracking the temporal and spatial changes in the IR and visible images in a specific identified ocular structure in real-time.

14. The method of claim 13, further comprising identifying the temporal and special changes of the specific identified ocular structure and utilizing the tracked OST temperatures as diagnostic indicators for ocular disease diagnosis and progression, regression, or remission.

15. The method of claim 13, wherein the specific identified ocular structures include pupil, iris, conjunctiva, and eyelids.

16. The method of claim 15, wherein the temporal and structural changes of the pupil, iris, conjunctiva, and eyelids are tracked over a period of time.

17. The method of claim 11, further comprising utilizing one or more digital processing modules to measure tear-film dynamic assessment and instability of the eye.

18. The system of claim 11, further comprising utilizing one or more digital processing modules to measure tear-film break-up time (TBUT).

19. The system of claim 11, further comprising utilizing one or more digital processing modules to measure and diagnose eye inflammation.

20. The system of claim 11, further comprising utilizing one or more digital processing modules to measure and diagnose dry eye.
